(19) United States
(12) Patent Application Publication
(10) Pub. No.: US 2013/0211305 A1
(43) Pub. Date: Aug. 15, 2013

(54) CHAFE FOR USE WITH ORTHOTIC BRACE AND LIKE OBJECTS

(71) Applicant: Kristin E. Heffernan, Chicago, IL (US)

(72) Inventor: Kristin E. Heffernan, Chicago, IL (US)

(73) Assignee: S&S Shapemaker, LLC, Hickory Hills, IL (US)

(21) Appl. No.: 13/579,525

(22) PCT Filed: Dec. 29, 2010
(86) PCT No.: PCT/US10/62287

§ 371 Date: Feb. 27, 2013

Related U.S. Application Data
(60) Provisional application No. 61/305,811, filed on Feb. 18, 2010.

(51) Int. Cl. A61F 5/01

(52) U.S. Cl. CPC A61F 5/0123
USPC 602/26

(57) ABSTRACT

A chafe for use with a leg brace or similar orthosis is disclosed. The chafe comprises a foundation element having a trough formation along one side, and a trough formation is adapted to accept a retainer loop. A top element fixedly overlies the foundation element and covers the retainer loop trough so as to retain the retainer loop within the trough and between the foundation element and the top element. The top element and/or a crown element has at least one ear formation adapted to selectively engage the retainer loop and restrict retainer loop rotation past a predetermined point.

This application claims priority from U.S. provisional patent application Ser. No. 61/305,811 filed Feb. 18, 2010 entitled Chafe for Use with Orthotic Brace and like Objects.

The present invention relates to a strap securing device, sometimes referred to as a chafe, for use with orthotic devices including leg braces and the like.

### BACKGROUND OF THE INVENTION

Braces and like orthoses are widely used in the correction of leg deformities, ankle injuries, and other conditions. One such orthosis is described and illustrated in Molino, et al. U.S. Pat. No. 7,147,612. The device illustrated therein includes a series of pivotally interconnected struts attached to tibial and femoral parts of the human leg by a series of straps. The straps are connected to the struts and to other parts of the brace by inter-connectors or fixtures known as chafes.

It is an object of the present invention to provide a chafe that can be easily manufactured and that is relatively inexpensive, but which, in use, permits the orthosis wearer to easily but properly and snugly secure an associated orthotic strap in a desired location upon the wearer's anatomy.

Other objects and advantages of the invention will become apparent upon reading the following detailed description and upon reference to the drawings. Throughout the drawings, like reference numerals refer to like parts.

### BRIEF DESCRIPTION OF THE DRAWINGS

FIG. 1 is a perspective view of an individual and a tibial leg brace.

FIG. 2 is a partial anatomical view of the tibial leg portion of the individual shown in FIG. 1 as the brace is being placed upon the wearer's leg.

FIG. 3 is a partial anatomical view similar to FIG. 2 showing the wearer's leg and the brace as the brace appears after it has been secured upon the wearer's leg.

FIG. 4 is an isometric view of the chafe of the present invention.

FIG. 5 is a top plan view of the chafe shown in FIG. 4.

FIG. 6 is a bottom plan view of the chafe shown in FIGS. 4 and 5.

FIG. 7 is a side elevational view of the chafe shown in FIGS. 4, 5 and 6.

FIG. 8 is a front elevational view of the chafe shown in FIGS. 4-7.

FIG. 9 is a rear elevational view of the chafe shown in FIGS. 4-8.

FIG. 10 is a side elevational view of an alternate embodiment of the invention.

FIG. 11 is a side elevational view similar to FIG. 10 but showing the invention in an alternate configuration.

FIG. 12 is a top plan view of the alternate embodiment of the invention.

FIG. 13 is a bottom plan view of the alternate embodiment of the invention.

FIG. 14 is an isometric view of the alternate embodiment of the invention.

FIG. 15 is another isometric view of the alternate embodiment of the invention.

### DETAILED DESCRIPTION

While the invention will be described in connection with a preferred embodiment, it will be understood that it is not intended to limit the invention to this embodiment. On the contrary, it is intended to cover all alternatives, modifications and equivalents as may be included within the spirit and scope of the invention as defined by the appended claims.

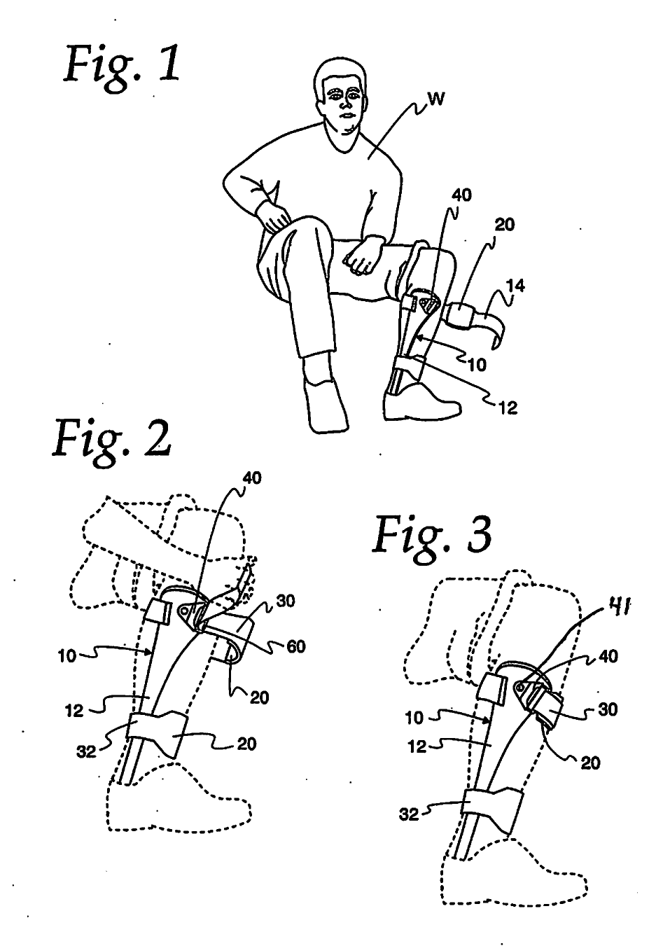

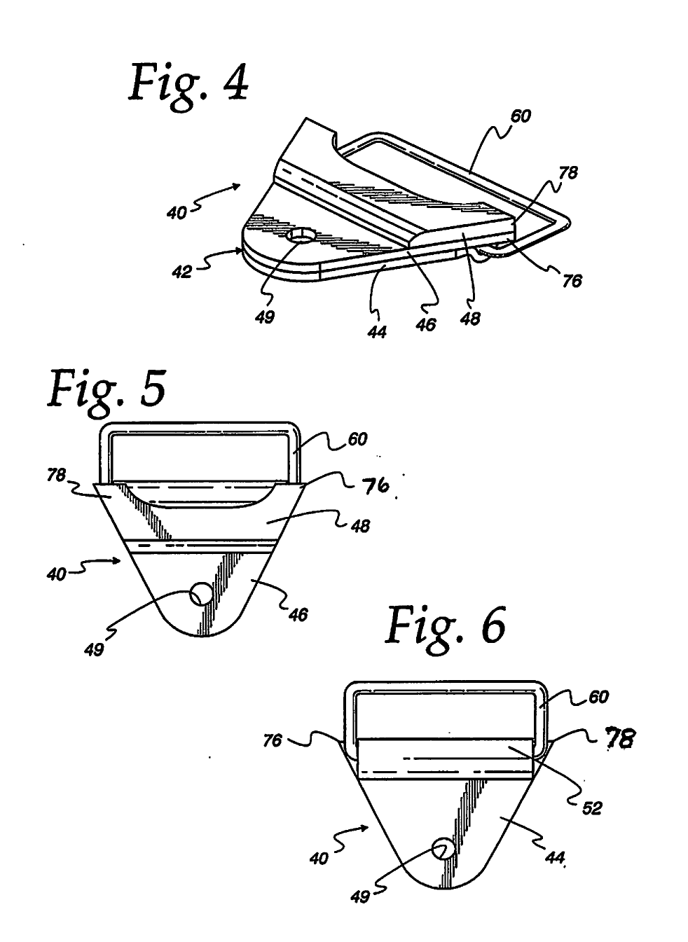

A leg brace 10 and a brace wearer W are illustrated in FIGS. 1-3. The brace 10 illustrated here includes one or more struts 12. These struts 12, pads 20, and other parts of the orthosis are held in desired positions adjacent to the wearer's leg by straps 30, 32. At least one strap 30 is attached to the brace strut 12 by a chafe 40. As shown in FIGS. 4-9, the chafe 40 illustrated comprises a body 42; an underlying foundation element 44; an overlying top element 46; and, if desired, a crown element 48. As shown in FIGS. 4, 5 and 6, a hole 49 can be provided through the elements 44 and 46 to permit the chafe 40 to be mounted to a brace strut 12 or other desired element by means of a rivet 41 (FIG. 3) or other convenient securing device.

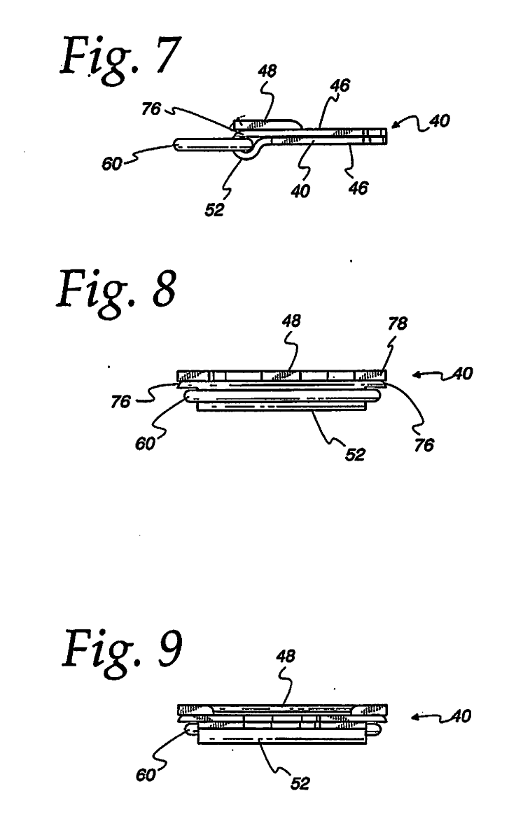

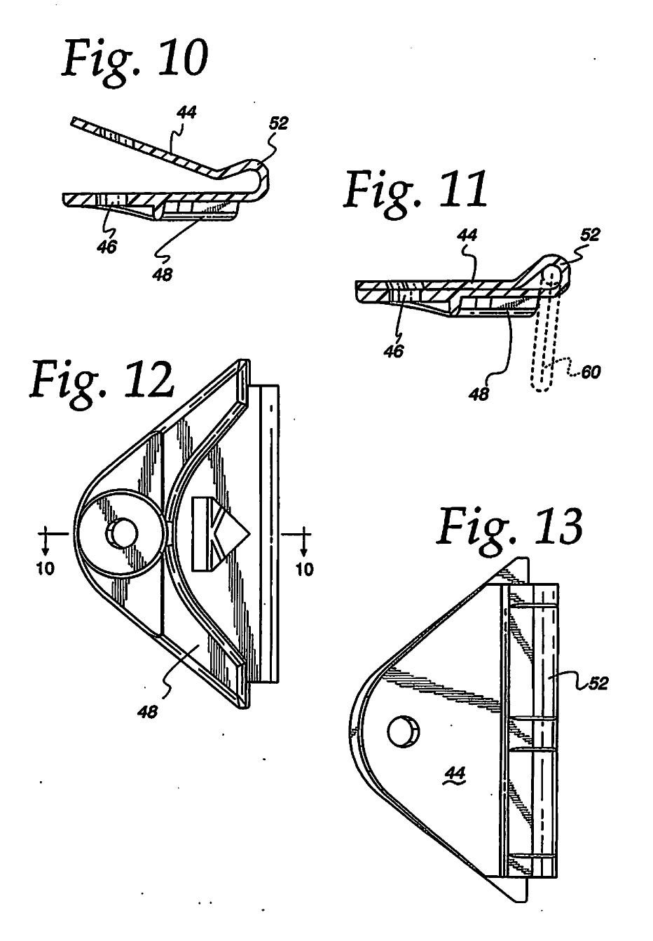

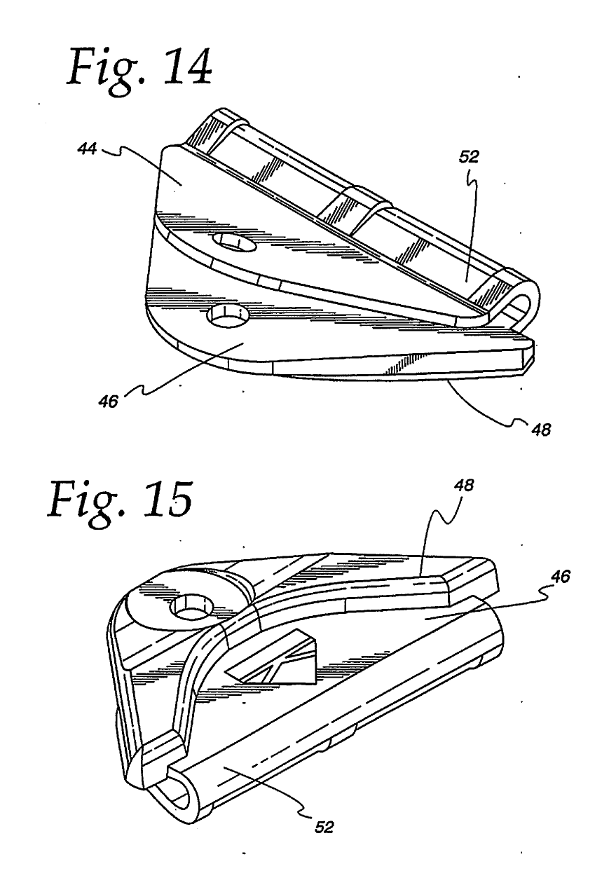

As particularly shown in FIGS. 6 and 7, the underlying element 44 is formed, molded, or otherwise provided with a trough-like formation 52 adapted to receive a rectangular strap-engaging retainer ring or loop 60. During chafe manufacture, the installed loop 60 and the trough-like formation 52 are covered by the top element 46 so as to retain the installed portion of the loop 60 in the trough 52.

Alternatively, the foundation element 44 and overlying top element 46 can be formed as a single piece and folded over one another to form the assembly, as shown in FIG. 10.

It is an object of the present invention to provide a chafe which permits the orthosis wearer to easily but properly and snugly secure an orthotic strap associated with the chafe in a desired position upon the wearer's anatomy. In accordance with this object, the trough 52 and the loop 60 are mutually sized so as to permit the loop 60 to swing or pivot freely with respect to the other elements of the chafe. However, the overlying element 46 and, if desired, the crown element 48 are provided with outwardly and laterally extending ears 76, 78, respectively. As shown in FIGS. 4, 5 and 6, the ears 76 are located so as to engage the underlying portions of the loop 60 and stop or prevent pivoting motion or rotation of the loop 60 past a loop position which extends outwardly and freely but roughly parallel to the orientation of the foundation element 44 and overlying top element 46. If desired, the crown element 48 alone can be provided with these loop-rotation stop ears 76, 78. The retainer loop 60 will not fold back over the top element 46 into a difficult-to-reach or difficult-to-manipulate position. This permits the brace wearer to preliminarily put the brace in a desired position on, for example, his leg as shown in FIG. 2 and then pull a strap 30 through the properly oriented and easily engaged loop 60. If the strap 30 is provided with appropriate hook-and-loop materials such as VELCRO hook and loop material, the strap may be secured in place on the leg with just one of the wearer's hands. Another hand may not be needed to maintain the loop 60 in a desired freely accessible location as the strap 30 is being threaded through the loop 60 and thereafter pulled snugly into a desired position. VELCRO is a trademark of Velcro USA Inc. of Manchester N.H.

The chafe elements 44, 46 and 48 may be made from any desired material having sufficient integral strength and having a commercially attractive price and availability. The elements may be cut or otherwise formed from one-eighth inch thick polypropylene, for example; and the elements may be secured to one another by a rivet or other known means. It will be noted that all the elements 44, 46, 48 and 60 are formed with relatively rounded corners and beveled edges where necessary to discourage these elements and the chafe itself from catching on the wearer's clothes.

The following is claimed as invention:

1. A chafe comprising a foundation element, having a trough formation along one side, the trough formation adapted to accept a retainer loop;
a top element fixedly overlying the foundation element and covering the retainer loop trough so as to retain the retainer loop within the trough and between the foundation element and the top element; and
at least one ear formation affixed to at least one element, the ear formation adapted to selectively engage the retainer loop and restrict retainer loop rotation past a predetermined point.

2. A chafe according claim 1 including a crown element affixed to one of said top element or said foundation element, said crown element being provided with said ear formation.

3. A chafe according to claim 1 wherein at least one of said elements is formed of polypropylene, polyethylene and mixtures thereof.

4. A chafe according to claim 1 wherein at least two of said elements are affixed to one another by an interconnecting fastener.

5. A chafe according to claim 4, wherein the loop is rectangular in shape.

6. A method of putting on a leg brace by the person who will wear the brace. unaided by any other person, the brace having a chafe mounting a strap and a loop and ear means for restricting the rotation of the loop with respect to a chafe body,
the method comprising the steps of
preliminarily putting the brace and a desired position with one hand,
putting the brace strap free end portion through the chafe loop with the other hand, and securing the strap free end portion in a position to secure the brace in a desired position on the wearer's body.

7. A method according to claim 6 wherein the step of securing the strap free end portion includes the step of securing the strap free end portion to a mediate portion of said strap by a hook-and-loop connection.
